(19) United States
(12) Patent Application Publication
(10) Pub. No.: US 2018/0139931 A1
(43) Pub. Date: May 24, 2018

(54) STATE DETECTOR, METHOD OF USING STATE DETECTOR, AND STATE DETECTION SYSTEM

(71) Applicant: KYOCERA CORPORATION, Kyoto-shi, Kyoto (JP)

(72) Inventors: Kenichi MATSUI, Yokohama-shi, Kanagawa (JP); Tomoyuki TOUGASAKI, Sagamihara-shi, Kanagawa (JP)

(73) Assignee: KYOCERA Corporation, Kyoto-shi, Kyoto (JP)

(21) Appl. No.: 15/568,387

(22) PCT Filed: Apr. 14, 2016
(86) PCT No.: PCT/JP2016/002030

§ 371 Date: Oct. 20, 2017

(30) Foreign Application Priority Data
Apr. 28, 2015 (JP) 2015-091576

(51) Int. Cl. A01K 29/00; A61D 17/00

(52) U.S. Cl. CPC A01K 29/005; A61B 5/01; A61D 17/002

(57) ABSTRACT

A state detector is configured to detect a biological state of a domestic animal based on biological data detected by a biological sensor portion, the biological sensor portion being non-invasively attached to and facing a body surface of the domestic animal to detect the biological data.

### CROSS REFERENCE TO RELATED APPLICATION

This application claims priority to and the benefit of Japanese Patent Application No. 2015-091576 (filed on Apr. 28, 2015), the entire disclosure of which is incorporated herein by reference.

### TECHNICAL FIELD

The present disclosure relates to detecting the state of a domestic animal and the like.

### BACKGROUND

There is conventionally known a system for notification of parturition by transmitting information from a thermometer inserted in a cow's vagina, together with the cow's ID.

There is also conventionally known a system for notification of estrus by transmitting information from a thermometer inserted in a cow's vagina, together with the cow's ID.

Detecting the lying state and standing state of a cow in estrus using a temperature sensor which detects the temperature of a cow bed is also conventionally known.

### SUMMARY

A state detector is configured to detect a biological state of a domestic animal or the like based on biological data detected by a biological sensor portion, the biological sensor portion being non-invasively attached to and facing a body surface of the domestic animal to detect the biological data.

### BRIEF DESCRIPTION OF THE DRAWINGS

In the accompanying drawings:

FIG. 1 is a schematic view and partially enlarged view illustrating an example of attaching a state detector to a cow according to an embodiment;

FIG. 2 is a block diagram illustrating an example of the state detector;

FIG. 3 is a sectional view and surface view of a biological sensor portion of the state detector;

FIG. 4 is a sectional view of a body portion of the state detector;

FIG. 5 is a diagram schematically illustrating an example of measurements by a first biological sensor and a second biological sensor of the state detector; and

FIG. 6 is a diagram schematically illustrating an example of a screen of a user terminal receiving information from the state detector.

### DETAILED DESCRIPTION

The conventionally known systems require insertion of the thermometer into the cow's vagina. Also, according to the conventionally known detecting method, since the temperature sensor is installed in the cow bed, detection is not possible during grazing, and also the system is large-scale.

Embodiments of the disclosure are described below.

As illustrated in FIG. 1, a state detector 1 according to an example of the disclosure is a device for detecting estrus in, for example, a cow or a horse. In outline, the state detector 1 includes a body portion 100, a biological sensor portion 500, a signal portion 600, and a fixture 300 for fixing the body portion 100 to a tail.

The biological sensor portion 500 is fixed to, for example, an indented part at the underside of the cow's tail (from the tailhead to behind the buttocks), with tape, an adhesive, or the like. The signal portion 600 extends from the biological sensor portion 500 to the body portion 100. The signal portion 600 is circuitry for the exchange of signals and power between the biological sensor portion 500 and the body portion 100.

In this example, the body portion 100 is fixed to the topside of the tail using the fixture 300. The fixture 300 may be Velcro® (Velcro is a registered trademark in Japan, other countries, or both), a bandage, elastic tape (e.g. Acrylic Nylon Bandage by Aintree), or disposable tape. Use of these fixtures for wrapping of the legs or tails of riding horses is widely known. The body portion 100 also may be fixed by winding a band-like member. The body portion 100 may be manufactured so as to enable winding of a belt or a rope.

The signal portion 600 extends between a housing 504 of the biological sensor portion 500 and a housing 108 of the body portion 100, as illustrated in FIG. 1. The signal portion 600 may extend halfway around the cow's tail from the topside to the underside. A resin member having appropriate elasticity may be used for the signal portion 600.

The signal portion 600 includes various signal lines 601 for exchanging input and output signals between the body portion 100 and the biological sensor portion 500, and for supplying detection signals from a first biological sensor 501x and a second biological sensor 501y or required power to the body portion 100.

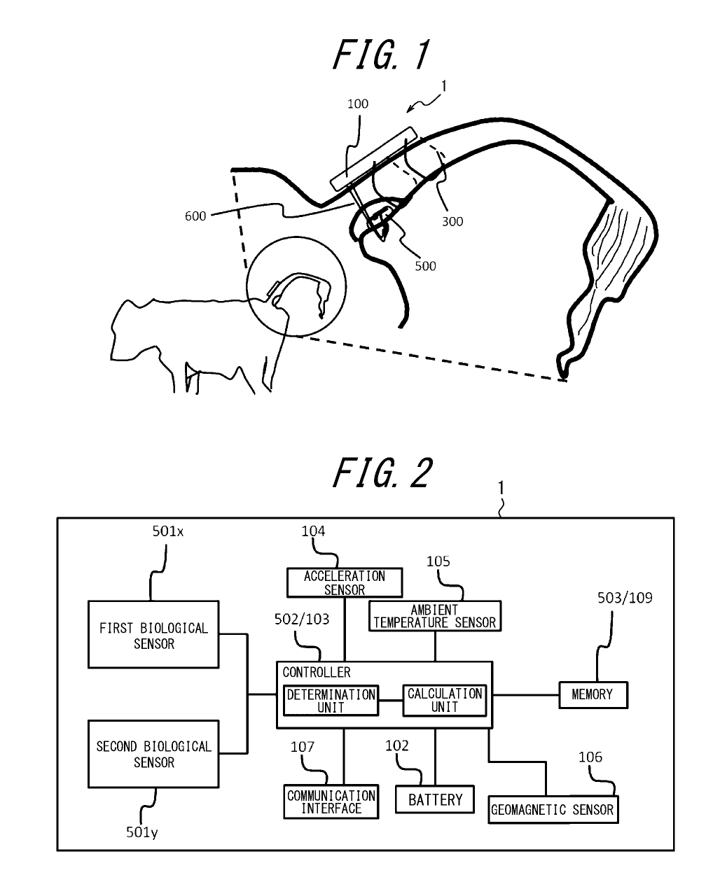

FIG. 2 is a block diagram illustrating an example of the state detector 1. A controller 502/103 performs various controls for the state detector 1. Here, the expression “controller (IC) 502/103” is used to refer to the case where the controller is included in the biological sensor portion 500 and the case where the controller is included in the body portion 100. In other words, the functions of the controller may be divided between the biological sensor portion 500 and the body portion 100, or the functions may all be included in one of the biological sensor portion 500 and the body portion 100. The controller 502/103 controls the first biological sensor 501x, the second biological sensor 501y, a battery 102, an acceleration sensor 104, an ambient temperature sensor 105, a geomagnetic sensor 106, a communication interface 107, and the like.

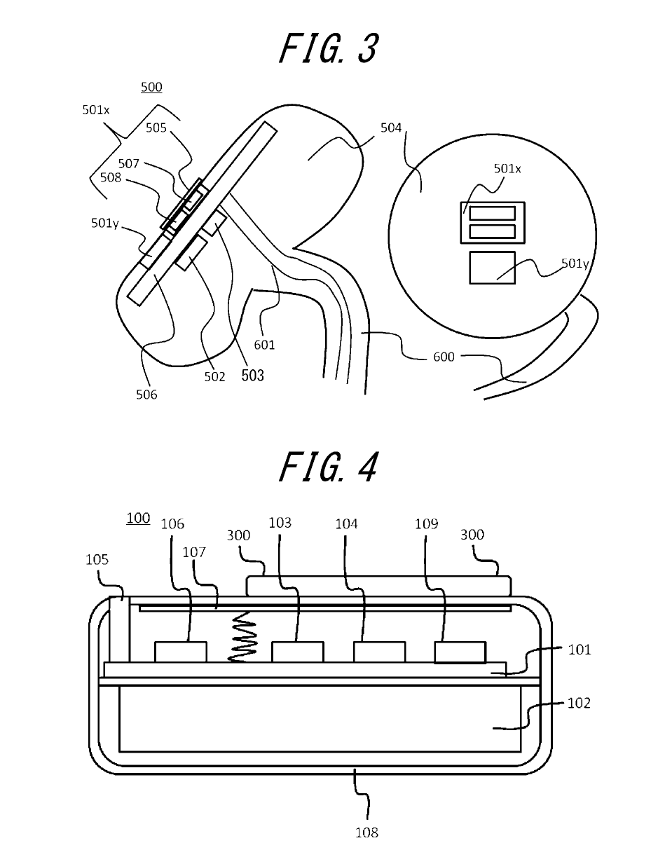

An example of the biological sensor portion 500 is now described with reference to FIGS. 2 and 3. The first biological sensor 501x in the biological sensor portion 500 detects, for example, the cow's pulse or a change in the blood flow in the cow's tail. The first biological sensor 501x in this embodiment is installed to face (i.e. front) a blood vessel from the underside of the cow's tailhead. In other words, a translucent panel 505 faces the blood vessel with the skin or soft tissue of the cow therebetween. An optical emitter 507 and an optical detector 508 are arranged in parallel in the housing 504 with a light-blocking wall therebetween. The biological sensor portion 500 has a structure in which the protective translucent panel 505 is positioned over the optical emitter 507 and the optical detector 508 to hermetically seal the housing 504.

The housing 504 may be made of hard resin such as polycarbonate or acrylic, or soft resin such as silicone rubber. Moreover, the housing 504 may be resin colored in black or the like, to prevent the passage of light around the optical emitter 507 and the optical detector 508. The dimensions of the external length and width of the housing 504 may be 3 cm or less, 2 cm or less, or 1 cm or less. Further, the housing 504 may be as thin as possible. For example, the thickness of the housing 504 may be 1 cm or less, 0.7 mm or less, or 0.4 mm or less. That is, the housing 504 may be as small as possible. Further, the housing 504 may also be as lightweight as possible. The weight of the housing 504 may be 100 g or less, 80 g or less, or 50 g or less.

For example, in the case of measuring the pulse of the cow, a light emitting diode (LED) or a laser, which emits blue light (wavelength: 400 nm to 430 nm) or green light (wavelength: 500 nm to 550 nm), is used as the optical emitter 507. The blue or green light associated with these wavelengths is easily absorbed by hemoglobin. When the blood flow is high, the absorption of light is high, and the output of the optical detector 508 is low. Alternatively, an LED or a laser, which emits red light (wavelength: 630 nm to 650 nm), may be used. In this case, because hemoglobin reflects infrared radiation, when the blood flow is high, the reflection of light is high, and the output of the optical detector is high. A photodiode corresponding to the respective wavelength of the optical emitter 507 is used as the optical detector 508.

In the case of measuring the blood flow, the state detector 1 uses, for example, a red (wavelength: 1.31 μm or 1.55 μm) laser to detect a relative blood flow from a phase difference in frequency caused by a Doppler shift.

The housing 504 includes a substrate 506 on which the controller 502 for controlling the emission timing, the emission intensity, the detection timing, etc. for pulse measurement is mounted. Although an example where the controller 502 is included in the biological sensor portion 500 is described here, the controller 502 need not necessarily be included in the biological sensor portion 500 as mentioned above. The controller 502 mounted on the substrate 506 not only controls the emission and detection timings of the optical emitter 507 and optical detector 508, but may also, for example, include a determination unit that determines an error or noise signal based on the signal from the optical detector 508, or a calculation unit that calculates the pulse. The sampling period is 0.005 second to 0.1 second. The determination unit determines an error has occurred in the event that a pulse with a frequency that is higher than usual for the domestic animal is detected. Moreover, for example, when the acceleration sensor 104 or the geomagnetic sensor 106 detects excessive movement of the cow or horse (or only its tail), the determination unit may determine that the measurement data is not accurate (an error) and reject the measurement data.

In the case where the first biological sensor 500x is a sensor for measuring blood flow, the first biological sensor 501x may detect relative blood flow from a phase difference in frequency caused by a Doppler shift using, for example, a red (wavelength: 1.31 μm or 1.55 μm) laser as the optical emitter 507. That is, the first biological sensor 501x acquires, as blood flow data, information regarding the blood flowing inside the living body, based on a Doppler shift. The first biological sensor 501x irradiates the blood flowing through the blood vessel with laser light from the optical emitter (i.e. laser optical emitter) 507. The first biological sensor 501x detects scattered light from the substance in the body, including scattered light from the blood, using the optical detector 508. The first biological sensor 501x calculates, as blood flow data, the blood velocity based on the difference in wavelength of scattered light from the blood (Doppler shift). The laser light emitted from the optical emitter 507 may be light with a wavelength of 1.31 μm, which has high transmittance through skin and low absorption in hemoglobin. The optical emitter 507 may be a distributed feedback laser that oscillates in a single longitudinal mode. In the case of detecting blood flow, the first biological sensor 501x may be a laser irradiation sensor, or an ultrasonic irradiation sensor that measures reflection by ultrasound.

The second biological sensor 501y measures, for example, the body temperature from the surface of the cow's tail. The second biological sensor 501y may be used to supplement the blood flow data of the first biological sensor 501x or determine an error.

In the case of measuring blood flow, as in the case of measuring the pulse, the controller 502 inside the housing 504 not only controls the emission timing and intensity and detection timing of the optical emitter 507 and optical detector 508, but also may remove any error or noise signal from the signal from the optical detector 508, or include a calculation unit that calculates the blood flow. The sampling period may be 0.005 seconds to 0.1 second.

In the case where one sensor for measuring the pulse and one sensor for measuring the body temperature are provided, the first biological sensor 501x may be a pulse sensor and the second biological sensor 501y may be a body temperature sensor, for example. Alternatively, these biological sensors may be a combination of a blood flow sensor and a pulse sensor, or a combination of a blood flow sensor and a body temperature sensor. A body temperature sensor includes an optical detector that detects infrared radiation from, for example, the blood vessel at the underside of the tail, to measure the body temperature. In the case where the biological sensor is a body temperature sensor, the substrate 506 and the controller 502 may be contained in the housing 504 as in the above-mentioned example. For example, the controller 502 mounted on the substrate 506 controls the operation of the optical detector for measuring the body temperature, and manages body temperature data.

A memory 503 stores biological data from each biological sensor and data from each sensor (such as the acceleration sensor and the geomagnetic sensor). Although the biological sensor portion 500, as disclosed in relation to FIG. 3, includes the memory 503, the body portion 100 may include a memory 109, or both the biological sensor portion 500 and the body portion 100 may include the respective memories. Biological data and information such as the calculated blood flow or pulse, and their respective error rates stored in the memory 503/109 may, when necessary, be provided to an external component (e.g. a user terminal such as a smartphone to which a software application for livestock estrus or health management has been downloaded, or a server of a manufacturer providing such an application) via the communication interface 107 or the like.

Next, the body portion 100 is described with reference to FIG. 4. The body portion 100 includes the housing 108 and, arranged inside the housing 108, a substrate 101, a power source (storage cell or dry cell) 102, the controller 103 (which may be omitted if the biological sensor portion 500 includes the controller 502), the acceleration sensor 104, the ambient temperature sensor 105, the geomagnetic sensor 106, the communication interface 107, and the memory 109. The fixture 300 is attached to the outside of the housing 108.

The substrate 101, the controller 103 mounted on the substrate 101, and the memory 109 have the same functions as those in the biological sensor portion 500 described above. That is, the controller 103 and the memory 109 may be used to control various functional components and electrical components in the biological sensor portion 500 and the body portion 100, and perform necessary calculations.

The acceleration sensor 104 may detect, for example, the cow or horse moving its tail to brush away insects. In this case, since the detection by the biological sensor is likely to be erroneous (false detection), when the acceleration sensor detects an acceleration equal to or greater than a predetermined acceleration, the measured value may be rejected. That is, the acceleration sensor 104 may be used to determine whether or not to reacquire data.

The ambient temperature sensor 105 is capable of detecting a phenomenon such as an abnormal increase or decrease of blood flow caused by, for example, extremely cold weather or an air temperature rise due to extremely hot weather. This eases the distinction between a change in pulse or blood flow due to estrus or a disease and a change in pulse or blood flow due to other external factors (weather factors). The data acquired by the ambient temperature sensor 105 may not necessarily enable such distinction, but may be used as supplementary data upon making the distinction.

The geomagnetic sensor 106 detects, for example, a rotational movement of the cow or horse. The data acquired by the geomagnetic sensor 106, together with the data acquired by the acceleration detector 104, enables detection of the cow's behavior, and thus can be used for error determination and the like. That is, the data acquired by the geomagnetic sensor 106 eases the determination of any abnormal behavior of the cow or horse, and so may be used by the controller to perform error determination and reject the detected value.

The communication interface 107 may use a conventionally known communication method. For example, the communication interface 107 may comply with a communication method such as Code Division Multiple Access (CDMA) or Long Term Evolution (LTE), or use Bluetooth or Wi-Fi. In the case where a micro base station for Bluetooth® (Bluetooth is a registered trademark in Japan, other countries, or both) or Wi-Fi® (Wi-Fi is a registered trademark in Japan, other countries, or both) can be installed in the cow bed, the use of Bluetooth or Wi-Fi saves more power than the use of a public wireless network such as CDMA or LTE.

While the disclosed devices, methods, and systems have been described by way of the drawings and embodiments, various changes or modifications may be easily made by those of ordinary skill in the art based on the present disclosure. Such various changes or modifications are therefore included in the scope of the present disclosure. For example, the functions included in the means, members, etc. may be rearranged without logical inconsistency, and a plurality of means, members, etc. may be combined into one means, member, etc. and a means, member, etc. may be divided into a plurality of means, members, etc.

Next, an example of using the state detector 1 in the case where the biological sensor portion 500 is a combination of a body temperature sensor and a blood flow sensor is described.

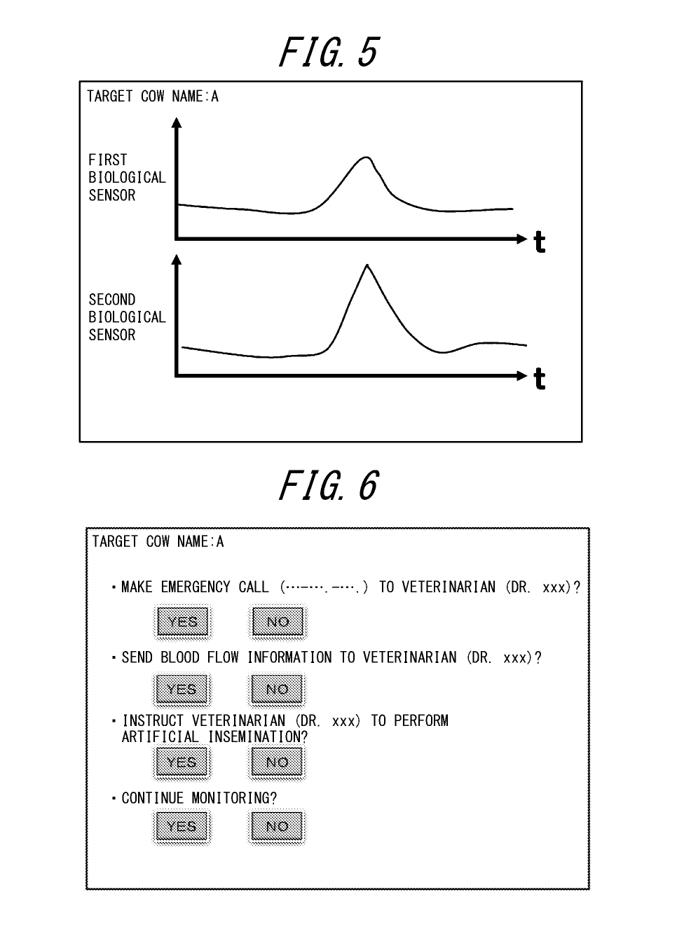

First, the sensor is powered on, and simultaneously or sequentially starts to measure blood flow and body temperature. Next, the biological sensor portion 500 acquires data. Next, in the case where the blood flow exceeds normal blood flow by, for example, more than 10%, the body portion 100 may notify the possibility of estrus or the like. In the case where supplementary body temperature data which is measured simultaneously also exhibits a predetermined increase, the body portion 100 can notify the possibility of estrus with greater accuracy. FIG. 5 schematically illustrates an example of the measurements of the first biological sensor (blood flow) and second biological sensor (body temperature) on the user terminal.

FIG. 6 illustrates an example of a screen on a user terminal for notification in the case where estrus is detected and for subsequent selection of a measure. Thus, the state detector 1 can be combined with the user terminal to constitute a state detection system for estrus and the like. FIG. 6 illustrates an example of a screen displayed on the terminal. The user can operate the state detector 1 to issue instructions through the screen displayed on the terminal, such as to contact a veterinarian or to continue monitoring.

1. A state detector configured to detect a biological state of a domestic animal based on biological data detected by a biological sensor portion, the biological sensor portion being non-invasively attached to and facing a body surface of the domestic animal to detect the biological data.

2. The state detector according to claim 1,
wherein the biological state of the domestic animal includes estrus, a disease associated with a fever, or a disease associated with a decrease in body temperature.

3. The state detector according to claim 1,
wherein the biological sensor portion is configured to measure one or more of pulse, blood flow, and body temperature.

4. The state detector according to claim 1,
wherein the biological sensor portion includes an optical emitter and an optical detector.

5. The state detector according to claim 4,
wherein the optical emitter is configured to emit LED light with a predetermined frequency or laser light with a predetermined frequency.

6. The state detector according to claim 1,
wherein the biological sensor portion is attached to an underside of a tail of the domestic animal to contact the domestic animal's skin on the underside.

7. The state detector according to claim 6, further comprising
a body portion configured to exchange a signal and/or power with the biological sensor portion, via a signal line.

8. The state detector according to claim 7,
wherein the body portion is placed on a side of the tail opposite to the biological sensor portion.

9. The state detector according to claim 1,
wherein the biological sensor portion includes at least two sensors each configured to measure biological data associated with any one of pulse, blood flow, and body temperature, and
the biological state is detected from the biological data measured by the at least two sensors.

10. The state detector according to claim 1, further comprising
a communication interface configured to transmit the biological data or the biological state detected from the biological data, to outside of the state detector.

11. A state detection system comprising:
the state detector according to claim 1; and
a user terminal configured to display the biological state.

12. A method of using a state detector configured to detect a biological state of a domestic animal based on biological data detected by a biological sensor portion, the biological sensor portion being non-invasively attached to and facing a body surface of the domestic animal to detect the biological data.
